(19) United States
(12) Patent Application Publication
(10) Pub. No.: US 2023/0101903 A1
(43) Pub. Date: Mar. 30, 2023

(54) COMPOUNDS USEFUL AS ANTI-VIRAL AGENTS

(71) Applicant: NATURE CRETE PHARMACEUTICALS PC, Heraklion (GR)

(72) Inventors: Elias CASTANAS, Heraklion (GR); George SOURVINOS, Chalandri (GR); Marilena KAMPA, Heraklion (GR); Christos LIONIS, Atsipopoulo Rethymnon (GR); Stergios PIRINTSOS, Rethymno (GR); Athanasios PANAGIOTOPOULOS, Heraklion (GR); Melpomeni TSELIOU, Heraklion (GR)

(21) Appl. No.: 17/799,055

(22) PCT Filed: Feb. 11, 2021
(86) PCT No.: PCT/EP2021/053389

§ 371 Date: Aug. 11, 2022

(30) Foreign Application Priority Data
Feb. 11, 2020 (GR) 20200100068
Feb. 17, 2020 (GB) 2002141.6

(51) Int. Cl. A61K 31/015; A61P 31/14

(52) U.S. Cl. CPC A61K 31/015; A61P 31/14

(57) ABSTRACT

The present invention relates to compounds of Formula (I) and compositions containing one or more compounds of Formula (I). The compounds and compositions are useful as anti-viral agents and may be used in the treatment or prevention of diseases caused by ribonucleic acid (RNA) viruses.

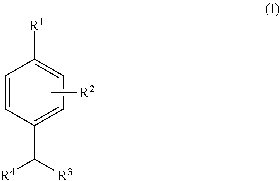

### FIELD OF THE INVENTION

The present invention relates to compounds useful as anti-viral agents. The invention also relates to compounds for use in the treatment or prevention of diseases caused by ribonucleic acid (RNA) viruses, and to methods of treating or preventing diseases caused by RNA viruses.

### BACKGROUND OF THE INVENTION

A viral disease is a disease that is caused by a virus, and may occur when an organism's body is invaded by a pathogenic virus. There are a multitude of viral diseases, including Ebola, SARS, SARS-CoV-2, rabies, common cold, hepatitis C, West Nile fever, poliomyelitis, measles and influenza.

Influenza is an infectious viral disease caused by an RNA virus of the orthomyxoviridae family. There are four genera of influenza viruses: A, B, C and D. Of these, influenza A causes the most serious symptoms; influenza B is less severe; and influenza C is usually only associated with minor symptoms. Influenza D is not known to infect humans.

Influenza A viruses are a global health concern, and have been responsible for several major pandemics that have killed many millions of people worldwide since 1900, such as the “Spanish flu” in 1918. Despite the significant effects influenza viruses can have on public health, known treatments for infections caused by influenza viruses still remain inadequate.

COVID-19 is a severe, mainly respiratory disease, caused by SARS-CoV-2, a novel coronavirus, isolated in late 2019, which caused a pandemic from early 2020 and is considered a major health issue by the WHO.

It would therefore be desirable to provide new compounds useful in treating viral diseases, in particular diseases caused by RNA viruses such as influenza or SARS-CoV-2.

### SUMMARY OF THE INVENTION

In one embodiment, the present invention provides a composition comprising one or more compounds of Formula (I), or a pharmaceutically acceptable salt, solvate, tautomer or isomer thereof, for use in the treatment or prevention of a disease caused by an RNA virus;

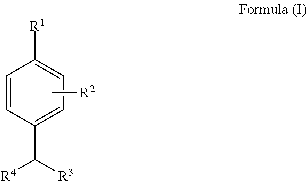

wherein:

R¹ is selected from —OH, C₁₋₆ alkyl which is optionally substituted by one or more —OH group, or C₁₋₆ alkoxy which is optionally substituted by one or more —OH group;

R² is selected from —H, halo, —NH₂, C₁₋₆ alkyl which is optionally substituted by one or more —OH and/or —NH₂ group, and a 5-membered heterocycle, which is optionally substituted by one or more C₁₋₆ alkyl group, each C₁₋₆ alkyl group being optionally substituted by one or more —OH and/or —NH₂ group;

R³ is selected from —C₁₋₆ alkyl which is optionally substituted by one or more —OH group; and

R⁴ is selected from —C₁₋₆ alkyl which is optionally substituted by one or more —OH group, and —C₁₋₆ alkylene-C₁₋₆ alkoxy which is optionally substituted by one or more —OH group.

In one aspect, the composition comprises at least about 30 wt. % of one or more compounds of Formula (I), or a pharmaceutically acceptable salt, solvate, tautomer or isomer thereof, for use in the treatment or prevention of a disease caused by an RNA virus, such as at least about 40 wt. %, 50 wt. %, 70 wt. %, 80 wt. %, 90 wt. %, 95 wt. % or 98 wt. %.

The present invention also provides a method of treating or preventing a disease caused by an RNA virus, using the above composition or a compound of Formula (I) or a pharmaceutically acceptable salt, solvate, tautomer or isomer thereof. In particular, the invention provides a method of treating or preventing a disease caused by an RNA virus, the method comprising administering to a human or non-human animal a therapeutically effective amount of the above composition or a compound of Formula (I) or a pharmaceutically acceptable salt, solvate, tautomer or isomer thereof.

The present invention also provides the above composition or a compound of Formula (I) or a pharmaceutically acceptable salt, solvate, tautomer or isomer thereof, for use in the manufacture of a medicament for the treatment or prevention of a disease caused by an RNA virus.

The present invention also provides a compound of Formula (I) for use in the treatment of SARS-CoV-2, preferably wherein the compound of Formula (I) is p-cymene.

### LIST OF FIGURES

FIG. 1 shows the effect of p-cymene on the activity of the H1N1 influenza virus compared with the known anti-viral drug Ribavirin.

FIG. 2 shows the effect of p-cymene on the concentration of intracellular influenza viral nucleoprotein.

FIG. 3 is a graph of influenza viral titer vs. concentration of an anti-viral compound of the invention after 24 hours.

FIG. 4 is a graph of influenza viral titer vs. concentration of an anti-viral compound of the invention after 48 hours.

FIG. 5 is a quantitative PCR analysis of newly synthesized influenza viral RNA of a cell treated with an anti-viral compound of the invention.

FIG. 6 presents a quantitative PCR analysis of SARS-CoV-2 newly synthesized viral RNA in the supernatant of infected VERO E6 cells, co-treated or pre-treated 2h before infection with different concentrations of p-cymene.

### DETAILED DESCRIPTION OF THE INVENTION

It has surprisingly been discovered that p-cymene and derivatives thereof have anti-viral activity against RNA viruses, and are therefore useful in the treatment or prevention of diseases caused by an RNA virus. p-Cymene is a naturally occurring aromatic organic compound having the formula set out below:

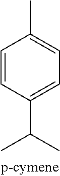

The present invention therefore provides a composition comprising one or more compounds of Formula (I), or a pharmaceutically acceptable salt, solvate, tautomer or isomer thereof, for use in the treatment or prevention of a disease caused by an RNA virus;

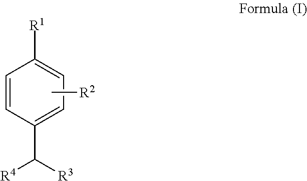

wherein:

R¹ is selected from —OH, C₁₋₆ alkyl which is optionally substituted by one or more —OH group, or C₁₋₆ alkoxy which is optionally substituted by one or more —OH group;

R² is selected from —H, halo, —NH₂, C₁₋₆ alkyl which is optionally substituted by one or more —OH and/or —NH₂ group, and a 5-membered heterocycle, which is optionally substituted by one or more C₁₋₆ alkyl group, each C₁₋₆ alkyl group being optionally substituted by one or more —OH and/or —NH₂ group;

R³ is selected from —C₁₋₆ alkyl which is optionally substituted by one or more —OH group; and

R⁴ is selected from —C₁₋₆ alkyl which is optionally substituted by one or more —OH group, and —C₁₋₆ alkylene-C₁₋₆ alkoxy which is optionally substituted by one or more —OH group.

Formula (I) therefore encompasses p-cymene and some derivatives thereof.

The composition preferably contains one or more compounds of Formula (I), wherein the total amount of the one or more compounds of Formula (I) is at least about 30 wt. %, such as at least about 40 wt. % or at least about 50 wt. %.

Preferably, the composition comprises at least about 60 wt. %, such as at least about 70 wt. %, of one or more compounds of Formula (I), based on the total weight of the composition.

More preferably, the composition comprises at least about 80 wt. %, such as at least about 90 wt. %, of one or more compounds of Formula (I).

Even more preferably, the composition comprises at least about 95 wt. %, such as at least about 98 wt. %, of one or more compounds of Formula (I).

In one embodiment, the composition consists essentially of one or more compounds of Formula (I). In another embodiment, the composition consists of one or more compounds of Formula (I).

In another embodiment, the composition may comprise a compound of Formula (I) in the amount of at least about 50 wt. %, such as at least about 60 wt. %, at least about 70 wt. %, at least about 80 wt. %, at least about 90 wt. %, at least about 95 wt. % or at least about 98 wt. %, based on the total weight of the composition. That is, a single compound of Formula (I) may constitute at least about 50 wt. % of the composition, such as at least about 60 wt. %, at least about 70 wt. %, at least about 80 wt. %, at least about 90 wt. %, at least about 95 wt. % or at least about 98 wt. %.

In one embodiment, the composition consists essentially of a compound of Formula (I). In another embodiment, the composition consists of a compound of Formula (I).

The composition may also comprise a pharmaceutically acceptable carrier.

Optionally, the composition contains a single compound of Formula (I), as defined above. Simply by way of example, the composition may comprise p-cymene but no other compound of Formula (I) as defined above.

Optionally, in any of the above embodiments, the compound of Formula (I) is not p-cymene. For example, optionally the composition does not include any p-cymene.

The present invention also provides a method of treating or preventing a disease caused by an RNA virus, the method comprising administering to a human or non-human animal a therapeutically effective amount of a compound of Formula (I) or a pharmaceutically acceptable salt, solvate, tautomer or isomer thereof.

The present invention also provides a compound of Formula (I) or a pharmaceutically acceptable salt, solvate, tautomer or isomer thereof, for use in the manufacture of a medicament for the treatment or prevention of a disease caused by an RNA virus.

The present invention also provides a composition comprising a compound of Formula (I) or a pharmaceutically acceptable salt, solvate, tautomer or isomer thereof, and optionally also a pharmaceutically acceptable carrier.

The present invention also provides a compound of Formula (I) or a pharmaceutically acceptable salt, solvate, tautomer or isomer thereof, for use in therapy.

The present invention also provides a composition comprising a compound of Formula (I) or a pharmaceutically acceptable salt, solvate, tautomer or isomer thereof, and optionally also a pharmaceutically acceptable carrier, for use in therapy.

The present invention also provides a composition comprising a compound of Formula (I) or a pharmaceutically acceptable salt, solvate, tautomer or isomer thereof, and optionally also a pharmaceutically acceptable carrier, for use in the treatment or prevention of a disease caused by an RNA virus.

### Definitions

As used herein, the term “C₁₋₆ alkyl” includes any saturated aliphatic hydrocarbon chains containing from 1 to 6 carbon atoms. Thus, C₁₋₆ alkyl includes any linear, branched or cyclic alkyl group as long as the group only contains from 1 to 6 carbon atoms. Suitable C₁₋₆ alkyl groups include methyl, ethyl, n-propyl, iso-propyl, cyclopropyl, n-butyl, iso-butyl, tert-butyl and cyclobutyl. Also included are alkyl groups containing a cyclic alkyl substituent, as long as the total number of carbon atoms is from 1 to 6, for example cyclopropyl, —CH₂-cyclopropyl, —CH₂CH₂-cyclopropyl or —CH₂CH₂CH₂-cyclopropyl.

Preferably, each C₁₋₆ alkyl group is a C₁₋₄ alkyl group (i.e. containing from 1 to 4 carbon atoms).

As used herein, the term “C₁₋₆ alkoxy” includes any C₁₋₆ alkyl group bonded through an oxygen group. Thus, the term “C₁₋₆ alkoxy” may be written as —O—C₁₋₆ alkyl, wherein “C₁₋₆ alkyl” is as defined above.

Preferably, each C₁₋₆ alkoxy group is a C₁₋₄ alkoxy group (i.e. containing from 1 to 4 carbon atoms).

Where indicated, each C₁₋₆ alkyl group and each C₁₋₆ alkoxy group may be substituted by one or more —OH group. Preferably, each C₁₋₆ alkyl group and each C₁₋₆ alkoxy group is substituted by at most one —OH group.

As used herein, the term “halo” refers to a halogen group, i.e. fluoro (F), chloro (CI), bromo (Br), or iodo (I). Preferred halo groups include F, CI and Br, with CI being most preferred.

As used herein, the term “5-membered heterocycle” refers to any 5-membered heterocyclic group, which may be saturated or unsaturated, and which may be aromatic. Examples of suitable 5-membered heterocyclic groups include imidazole, thiazole, pyrrole, oxazole, isoxazole, pyrazole, isothiazole and triazole.

Preferably, the 5-membered heterocycle contains at least one nitrogen atom, more preferably at least 2 nitrogen atoms and most preferably 3 nitrogen atoms. Most preferably, the 5-membered heterocycle is a triazole.

As used herein, the term “triazole” means 1,2,3-triazole or 1,2,4-triazole, preferably 1,2,4-triazole. More preferably, the 1,2,4-triazole is connected to the core aromatic ring via the 1N and any substituent is connected via the 3C, as shown below.

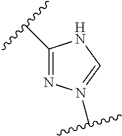

As used herein, the term “isomer” preferably means a stereoisomer, such as a diastereomer or an enantiomer. More preferably, the isomer is an enantiomer.

R¹

R¹ is selected from —OH, C₁₋₆ alkyl which is optionally substituted by one or more —OH group, or C₁₋₆ alkoxy which is optionally substituted by one or more —OH group.

Preferably, R¹ is selected from —OH, C₁₋₆ alkyl which is optionally substituted by —OH, or C₁₋₆ alkoxy which is optionally substituted by —OH.

More preferably, R¹ is selected from —OH, C₁₋₄ alkyl which is optionally substituted by —OH, and C₁₋₄ alkoxy which is optionally substituted by —OH.

Even more preferably, R¹ is selected from —OH, C₁₋₄ alkyl which is optionally substituted by —OH, and C₁₋₄ alkoxy which is optionally substituted by —OH.

Most preferably, R¹ is selected from —OH, —CH₃, —CH₂CH₃, CH₂OH, and —O—CH(OH)CH_{3.}

R²

R² is selected from —H, halo, —NH₂, 01-6 alkyl which is optionally substituted by one or more —OH and/or —NH₂ group, and a 5-membered heterocycle, which is optionally substituted by one or more C₁₋₆ alkyl group, each C₁₋₆ alkyl group being optionally substituted by one or more —OH and/or —NH₂ group.

Preferably, R² is selected from —H, —Cl, —NH₂, 01-6 alkyl which is optionally substituted by —OH and/or —NH₂, and triazole, which is optionally substituted by C₁₋₆ alkyl which is optionally substituted by —OH and/or —NH₂.

More preferably, R² is selected from —H, —Cl, —NH₂, 01-4 alkyl which is optionally substituted by —OH and/or NH₂, and triazole, which is substituted by C₁₋₄ alkyl substituted by —OH and/or NH₂.

Even more preferably, R² is selected from —H, —Cl, —NH₂, 01-2 alkyl which is optionally substituted by —OH and/or NH₂, and triazole, which is substituted by C₁₋₄ alkyl substituted by —OH and/or NH₂.

Most preferably, R² is selected from —H, —Cl, —NH₂, —CH₃, CH(OH)NH₂, and -triazole-CH(OH)(NH₂).

R³

R³ is selected from —C₁₋₆ alkyl which is optionally substituted by one or more —OH group.

Preferably, R³ is selected from —C₁₋₆ alkyl which is optionally substituted by —OH. More preferably, R³ is selected from —C₁₋₄ alkyl which is optionally substituted by —OH.

Even more preferably, R³ is selected from —C₁₋₂ alkyl which is optionally substituted by —OH.

Most preferably, R³ is selected from —CH₃, —CH₂CH₃ and —CH₂OH.

R⁴

R⁴ is selected from —C₁₋₆ alkyl which is optionally substituted by one or more —OH group, and —C₁₋₆ alkylene-C₁₋₆ alkoxy which is optionally substituted by one or more —OH group.

Preferably, R⁴ is selected from —C₁₋₆ alkyl which is optionally substituted by —OH, and —C₁₋₆ alkylene-C₁₋₆ alkoxy which is optionally substituted by —OH.

More preferably, R⁴ is selected from —C₁₋₄ alkyl which is optionally substituted by —OH, and —C₁₋₄ alkylene-C₁₋₄ alkoxy which is optionally substituted by —OH.

Even more preferably, R⁴ is selected from —C₁₋₂ alkyl which is optionally substituted by —OH, and —C₁₋₂ alkylene-C₁₋₄ alkoxy which is optionally substituted by —OH.

Most preferably, R⁴ is selected from —CH₂OH, —CH₂—O—CHs and —CH₂—O—CH₂CH₂CH₃.

Alternatively, R⁴ may be selected from —C₁₋₆ alkyl which is substituted by one or more —OH group, and —C₁₋₆ alkylene-C₁₋₆ alkoxy which is optionally substituted by one or more —OH group.

### Preferred Embodiments

Thus, in one embodiment of the invention: R¹ is selected from —OH, C₁₋₆ alkyl which is optionally substituted by —OH, or C₁₋₆ alkoxy which is optionally substituted by —OH;

R² is selected from —H, —CI, —NH₂, 01-6 alkyl which is optionally substituted by —OH and/or —NH₂, and triazole, which is optionally substituted by C₁₋₆ alkyl which is optionally substituted by —OH and/or —NH₂;

R³ is selected from —C₁₋₆ alkyl which is optionally substituted by —OH; and

R⁴ is selected from —C₁₋₆ alkyl which is optionally substituted by —OH, and —C₁₋₆ alkylene-C₁₋₆ alkoxy which is optionally substituted by —OH.

In another embodiment of the invention:

R¹ is selected from —OH, C₁₋₄ alkyl which is optionally substituted by —OH, and C₁₋₄ alkoxy which is optionally substituted by —OH;

R² is selected from —H, —CI, —NH₂, 01-4 alkyl which is optionally substituted by —OH and/or NH₂, and triazole, which is substituted by C₁₋₄alkyl substituted by —OH and/or NH₂;

R³ is selected from —C₁₋₄ alkyl which is optionally substituted by —OH; and

R⁴ is selected from —C₁₋₄ alkyl which is optionally substituted by —OH, and —C₁₋₄ alkylene-C₁₋₄ alkoxy which is optionally substituted by —OH.

In another embodiment of the invention:

R¹ is selected from —OH, C₁₋₄ alkyl which is optionally substituted by —OH, and C₁₋₄ alkoxy which is optionally substituted by —OH;

R² is selected from —H, —CI, —NH₂, 01-2 alkyl which is optionally substituted by —OH and/or NH₂, and triazole, which is substituted by C₁₋₄ alkyl substituted by —OH and/or NH₂;

R³ is selected from —C₁₋₂ alkyl which is optionally substituted by —OH; and

R⁴ is selected from —C₁₋₂ alkyl which is optionally substituted by —OH, and —C₁₋₂ alkylene-C₁₋₄ alkoxy which is optionally substituted by —OH.

In another embodiment of the invention:

R¹ is selected from —OH, —CH₃, —CH₂CH₃, CH₂OH, and —O—CH(OH)CH₃; R² is selected from —H, —CI, —NH₂, —CH₃, CH(OH)NH₂, and -triazole-CH(OH)(NH₂);

R³ is selected from —CH₃, —CH₂CH₃ and —CH₂OH; and R⁴ is selected from —CH₂OH, —CH₂—O—CH₃ and —CH₂—O—CH₂CH₂CH₃.

In another embodiment of the invention:

R¹ is selected from —OH, —CH₃, —CH₂CH₃ and CH₂OH;

R² is selected from —H, —CH₃ and CH(OH)NH₂;

R³ is selected from —CH₃, —CH₂CH₃ and —CH₂OH; and

R⁴ is selected from —CH₂—O—CH₃ and —CH₂—O—CH₂CH₂CH₃.

Alternatively, in any of the above embodiments, R⁴ may not be an unsubstituted alkyl group.

In another embodiment, the one or more compounds of Formula (I) are selected from the group consisting of:

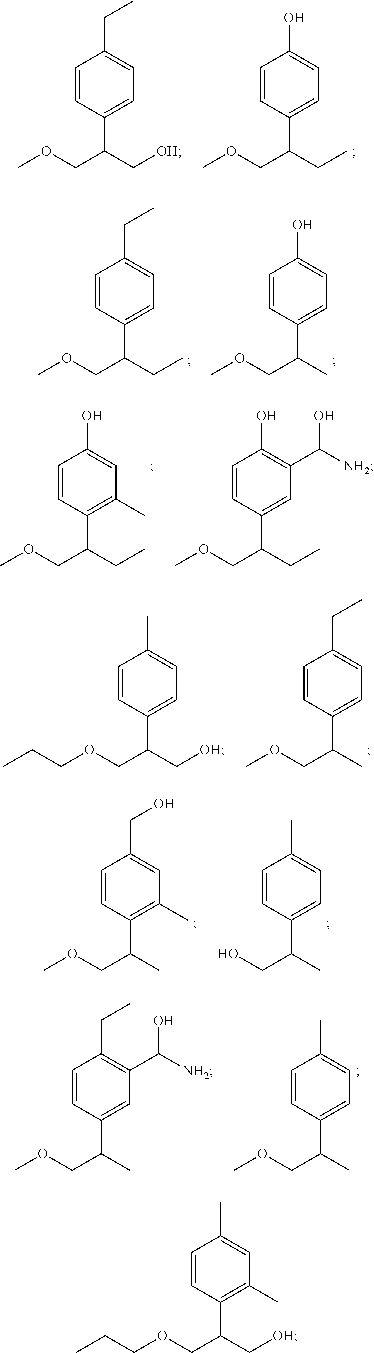

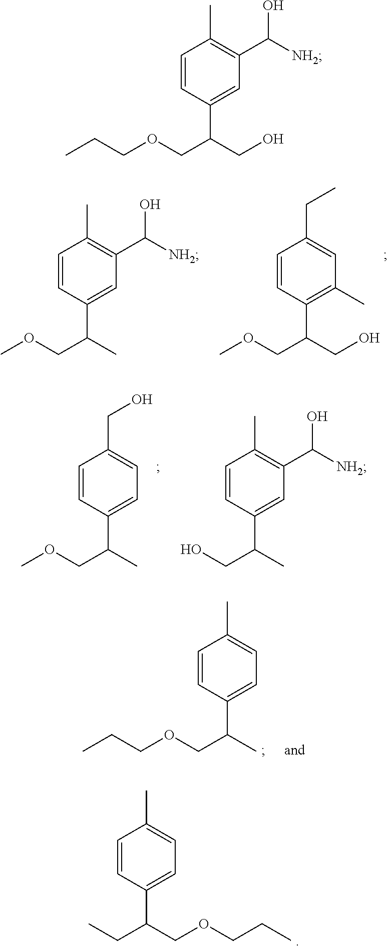

Most preferably, the one or more compounds of Formula (I) are selected from the group consisting of:

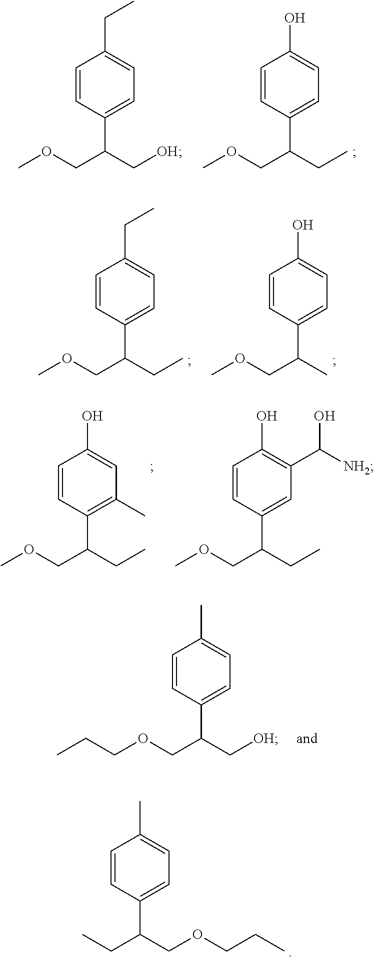

In each of the embodiments described above, the composition preferably comprises only one compound of Formula (I).

In another embodiment, the invention provides a composition comprising one or more compounds of Formula (I), or a pharmaceutically acceptable salt, solvate, tautomer or isomer thereof, for use in the treatment or prevention of a disease caused by an RNA virus;

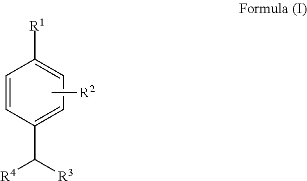

wherein:

R¹ is selected from —OH, C₁₋₆ alkyl which is optionally substituted by one or more —OH group, or C₁₋₆ alkoxy which is optionally substituted by one or more —OH group;

R² is selected from —H, halo, —OH, —NH₂, C₁₋₆ alkyl which is optionally substituted by one or more —OH and/or —NH₂ group, and a 5-membered heterocycle, which is optionally substituted by one or more C₁₋₆ alkyl group, each C₁₋₆ alkyl group being optionally substituted by one or more —OH and/or —NH₂ group;

R³ is selected from —C₁₋₆ alkyl which is optionally substituted by one or more —OH group; and R⁴ is selected from —C₁₋₆ alkyl which is optionally substituted by one or more —OH group, and —C₁₋₆ alkylene-C₁₋₆ alkoxy which is optionally substituted by one or more —OH group;

wherein the one or more compounds of Formula (I) are not p-cymene.

Thus, in one embodiment the composition does not comprise p-cymene.

RNA Viruses

An RNA virus is a virus that has ribonucleic acid (RNA) as its genetic material. This nucleic acid is usually single-stranded RNA (ssRNA), but may be double-stranded RNA (dsRNA). Examples of human diseases caused by RNA viruses include Ebola hemorrhagic fever (Ebola); respiratory diseases caused by coronaviruses, such as severe acute respiratory syndrome (SARS) and SARS-CoV-2 (also called 2019-nCoV acute respiratory disease, and caused by the novel 2019-nCoV Wuhan, COVID-19); rabies, common cold; influenza; hepatitis C; West Nile fever; poliomyelitis; and measles.

As used herein, the term “disease(s) caused by an RNA virus” includes any disease that is caused by a virus having RNA as its generic material. For example, the term “disease(s) caused by an RNA virus” may include any disease caused by a virus belonging to Group III, Group IV or Group V of the Baltimore classification system, preferably Group V.

Preferably, the term “disease(s) caused by an RNA virus” includes diseases selected from Ebola, respiratory diseases caused by coronaviruses (preferably selected from SARS and SARS-CoV-2), rabies, common cold, influenza, hepatitis C, West Nile fever, poliomyelitis and measles. More preferably, the disease is selected from COVID-19 (2019-CoV Wuhan), Ebola, rabies and influenza. Most preferably, the disease is influenza or COVID-19.

As used herein, the term “influenza” includes a disease caused by influenza A, B, C or D, preferably influenza A, B or C, more preferably influenza A or B, and most preferably influenza A.

RNA-viruses, including Influenza or SARS-CoV-2 replication occur in living cells in several stages, including (i) entry into the host cell, (ii) entry of viral ribonucleoproteins (vRNPs) into the nucleus, (iii) transcription and replication of the viral genome, (iv) export of the vRNPs from the nucleus; and (v) assembly and budding at the host cell plasma membrane (e.g. by rupture of the cell).

Some RNA viruses such as influenza, rabies, coronaviruses or Ebola form a nucleoprotein-RNA complex and act as a scaffold for nucleocapsid formation and as a template for RNA replication and transcription by condensing RNA into the virion, providing a mechanism for pharmaceutical intervention.

Without wishing to be bound by theory, it is believed that the compounds of the present invention inhibit the reproduction of RNA viruses by binding to viral nucleoprotein or nucleocapsid (NC) protein, thereby inhibiting its interaction with RNA. This prevents the nucleoprotein/NC-RNA complex from forming, thereby resulting in a decrease in viral RNA reproduction and hence resulting in a decrease in production of new viral capsids. Alternatively, compounds may interact with the Nuclear Localization Signal (NLS) of the protein, inhibiting its recognition by karyopherins, which are responsible for the nuclear transportation of the nucleoprotein/NC-vRNA complex.

RNA viruses generally have high mutation rates compared to DNA viruses, because viral RNA polymerases lack the proof-reading ability of DNA polymerases. This is a major reason for the difficulty in developing effective vaccines to prevent diseases caused by RNA viruses.

In contrast, nucleoproteins (NPs) or NCs are conserved molecules among a few RNA viruses and are not subject to mutations. There is also no cellular equivalent protein. This makes them good targets for anti-viral agents, since any compound which can inhibit binding of the nucleoprotein to the RNA will act even if there is a mutation of the RNA.

The compounds of Formula (I) may be administered in any manner conventional for the treatment of the indicated diseases, including but not limited to orally, parenterally, sublingually, transdermally, rectally, via inhalation or via buccal administration.

The compounds of Formula (I) may be administered alone or in combination. The compound(s) may also be administered as part of a composition comprising one or more compounds of Formula (I) and a pharmaceutically acceptable carrier.

The pharmaceutically acceptable carrier may be any carrier known in the art for the delivery of biologically active agents to animals such as humans. The pharmaceutically acceptable carrier may be, for example, either a solid or a liquid. Suitable pharmaceutically acceptable carriers would be known to those skilled in the art.

By way of example, suitable solid pharmaceutically acceptable carriers include lactose, terra alba, sucrose, talc, gelatin, agar, pectin, acacia, magnesium stearate, stearic acid, microcrystalline cellulose, polymer hydrogels and the like. Suitable pharmaceutically acceptable liquid carriers include propylene glycol, peanut oil, olive oil and the like. Any person skilled in the art may extend this list to any suitable carrier, related to the mode of administration.

The dosage regimen for the compounds and compositions of the present invention will, of course, vary depending upon factors such as the age, sex, health, medical condition and weight of the recipient; the nature and extent of the symptoms; the nature of any concurrent treatment; the frequency of treatment; and the route of administration. In particular, it is noted that compounds and compositions of the present invention may be formulated for use in therapy, or for use as a prophylactic.

The compounds of Formula (I) may be used in any amount which is non-toxic and which is sufficient to produce the desired pharmacological activity in the human or non-human animal concerned. Suitable doses for administration may range from about 1 to about 1000 μg/kg, for example from about 10 to about 100 μg/kg or from about 30 to about 60 μg/kg. These doses may be administered from 1 to 6 times per day, for example from 2 to 4 times per day.

Compounds Per Se

The present invention is also directed to compounds of Formula (I) or pharmaceutically acceptable salts, solvates, tautomers or isomers thereof selected from the group consisting of:

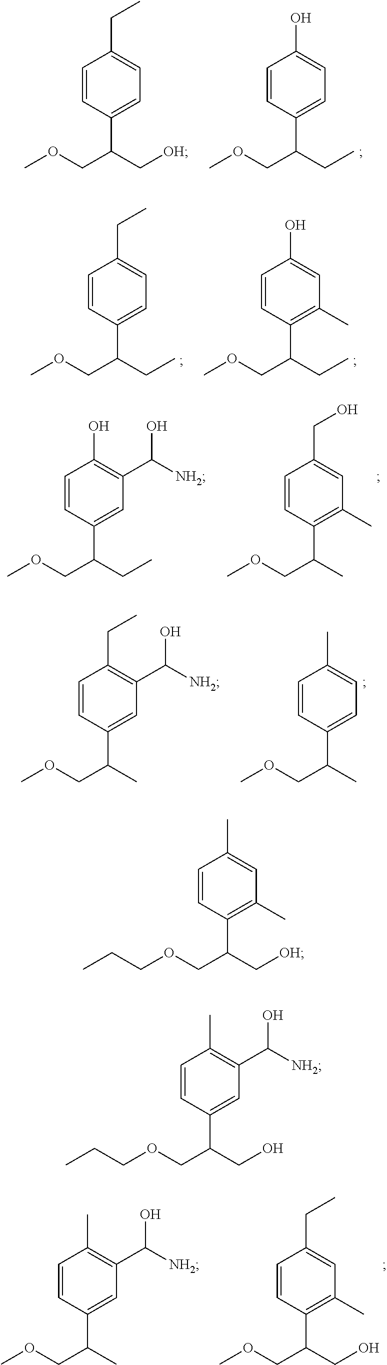

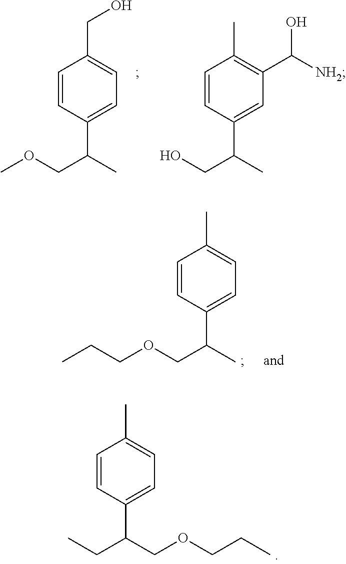

In another embodiment, the present invention is directed to a compound of Formula (I) selected from the group consisting of:

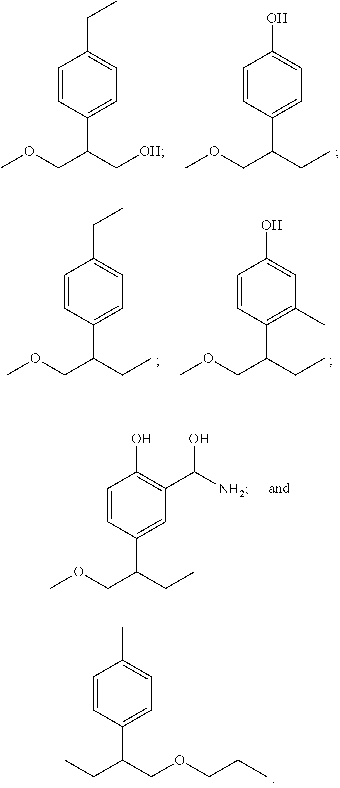

Compounds listed in the two groups set out above will be referred to herein as compounds of Formula (Ia).

The present invention also provides a composition comprising one or more compounds of Formula (Ia) or a pharmaceutically acceptable salt, solvate, tautomer or isomer thereof, and optionally also a pharmaceutically acceptable carrier.

In said composition the one or more compounds (preferably one compound) of Formula (Ia) may be present in the amount of at least about 50 wt. %, such as at least about 60 wt. %, at least about 70 wt. %, at least about 80 wt. %, at least about 90 wt. %, at least about 95 wt. % or at least about 98 wt. %, based on the total weight of the composition.

In one embodiment, the composition may consist essentially of one or more compounds (preferably one compound) of Formula (Ia). In another embodiment, the composition may consist of one or more compounds (preferably one compound) of Formula (Ia).

The present invention also provides a compound of Formula (Ia) or a pharmaceutically acceptable salt, solvate, tautomer or isomer thereof, for use in therapy.

The present invention also provides a composition comprising one or more compounds of Formula (Ia) or a pharmaceutically acceptable salt, solvate, tautomer or isomer thereof, and optionally also a pharmaceutically acceptable carrier, for use in therapy.

The present invention also provides a compound of Formula (Ia) or a pharmaceutically acceptable salt, solvate, tautomer or isomer thereof, for use in the treatment or prevention of a disease caused by an RNA virus.

The present invention also provides a composition comprising one or more compounds of Formula (Ia) or a pharmaceutically acceptable salt, solvate, tautomer or isomer thereof, and optionally a pharmaceutically acceptable carrier, for use in the treatment or prevention of a disease caused by an RNA virus.

The present invention also provides a method of treating or preventing a disease caused by an RNA virus, the method comprising administering to a human or non-human animal a therapeutically effective amount of a compound of Formula (Ia) or a pharmaceutically acceptable salt, solvate, tautomer or isomer thereof.

The present invention also provides a compound of Formula (Ia) or a pharmaceutically acceptable salt, solvate, tautomer or isomer thereof, for use in the manufacture of a medicament for the treatment or prevention of a disease caused by an RNA virus.

Synthesis of Compounds of Formula (I)

A person skilled in the art of organic chemistry would understand how to synthesis compounds of Formula (I) using their common general knowledge. Nevertheless, Schemes 1 and 2 are provided below showing examples of how the compounds of Formula (I) could be made. These schemes are not intended to be limiting, and other methods of synthesising the compounds of Formula (I) may be available.

In Scheme 1, Compounds 2a and 1c are reacted together to form a compound of Formula (I). Compound 2a may be commercially available, or may be synthesised from a commercially available precursor using routine synthesis known to those skilled in the art.

Similarly, Compound 1c may also be commercially available. However, in Scheme 1 Compound 1c is shown as being synthesised using known synthetic steps from Compounds 1 b and 1a in turn, each of which may be commercially available.

Suitable conditions for the synthetic steps shown in Schemes 1 and 2 would be known to those skilled in the art. However, by way of example, Compound 1a may be reacted with NaN₃ and NaNO₂ at low temperatures (e.g. less than 0° C.) to form Compound 1 b, whilst Compound 1 b may be reacted with KI to form Compound 1c. Compounds 1c and 2a may be reacted together under any conditions suitable for a Suzuki reaction, which would be well known to the skilled person. For example, a palladium catalyst may be used in basic conditions.

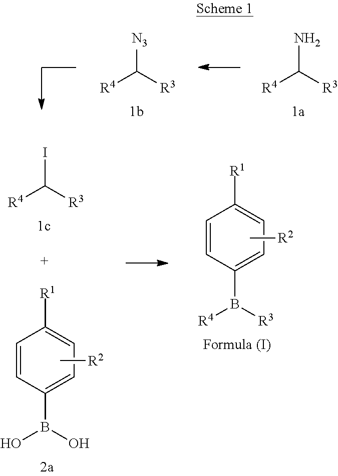

In another possible synthesis, the substituent R⁴ is added last. One example of such a synthesis where R⁴ is —CH₂—O—CH₂CH₂CH₃ is shown in Scheme 2.

Compound 1c′ as shown in Scheme 2 may be commercially available, or it may be synthesised. For example, Compound 1c′ may be synthesised in the same was as shown in Scheme 1 in relation to Compound 1c. Compounds 1c′ and 2a may also be reacted together under any conditions suitable for a Suzuki reaction, which would be well known to the skilled person.

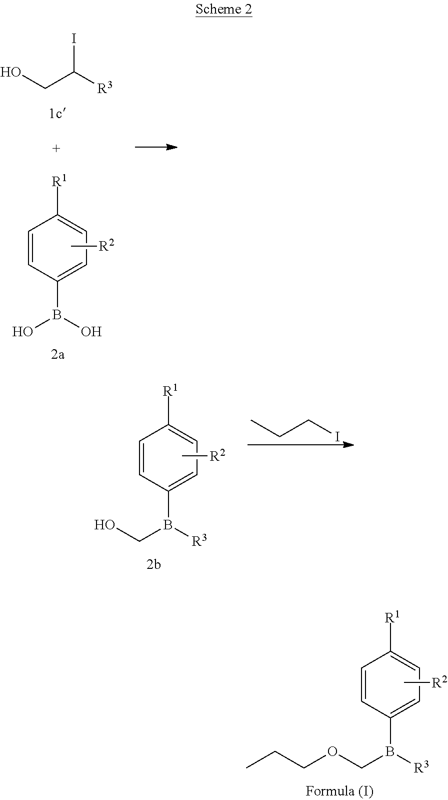

### Examples

All assays in Examples 1, 2, 4 and 5 were performed with the human Influenza A/FM/1/47/H1N1 strain, and canine MDCK-II cells, whilst in Example 7 VERO E6 monkey kidney cells were used.

The MDCK-II (ATCC® CCL34™) cells were grown according to standard conditions, as described in Glatthaar-Saalmüller et al., Phytomedicine, 2015, 22(10), pp911-920, while VERO E6 (ATCC® CRL-1586) were cultured in DMEM, 10% fetal bovine serum (FBS), with antibiotics, at 37° C., 5% CO₂.

The influenza A/FM/1/47/H1N1 virus was obtained from American Type Culture Collection (ATCC, Manassas Va., USA), while SARS—CoV-2 (isolate 30-287) was obtained through culture in Vero E6 cells, from an infected patient, in Alexandroupolis, Greece.

### Example 1-p-Cymene Anti-Viral Activity

The antiviral activity of p-cymene in Influenza A/FM/1/47/H1N1 was assayed in a plaque-reduction assay (expressed in forming plaques).

Canine MDCK-II cells were seeded into 6-well plastic plates and cultured at 37° C. under 5% CO₂ for 24 hours. The MDCK-II cells were then inoculated with Influenza A/FM/1/47/H1N1 for 2 hours, after which the infected cells were washed and cultured for 72 hours at 37° C. under 5% CO₂ in the presence of different concentrations of p-cymene in Minimum Essential Medium (MEM). Ribavirin was used as a control at a concentration of 25 μM.

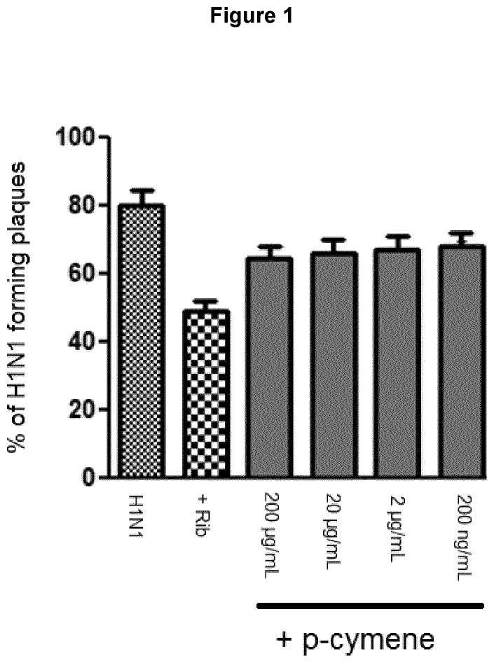

After incubation, the virus-infected cells were observed microscopically. After 3 days of infection, cells were fixed with paraformaldehyde and cell monolayers were stained with crystal violet. FIG. 1 shows the number of forming plaques (%) for the different concentrations of p-cymene and ribavirin (control).

As can be seen from FIG. 1, p-cymene induced a significant, dose-dependent decrease of plaque formation at all concentrations above 200 ng/mL.

### Example 2-p-Cymene Anti-Viral Activity

Intracellular viral nucleoprotein (NP) concentration was quantified by Western Blot Analysis at 7, 10 and 24 hours after infection.

A six-well culture plate was seeded with cells, which were then incubated at 37° C. for 24 hours under 5% CO₂. The cells were infected with influenza virus A/FM/1/47 at 37° C. for 1 hour and then p-cymene was added (200 ng/mL). The cells were harvested at the indicated time periods (7h, 10h and 24h). The lysates were incubated for 20 min on ice and then centrifuged at 14,000×g for 10 min at 4° C. The supernatants were collected and equal amounts of proteins were resolved by sodium dodecyl sulfate (10%) polyacrylamide gel electrophoresis.

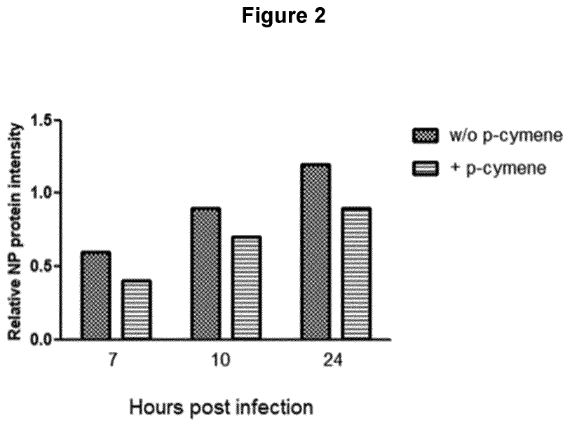

The results are shown in FIG. 2.

Influenza A nucleoprotein (NP) has a fundamental role for the establishment of the successful viral infection, being the major component of the viral nucleoprotein complex and regulating vRNP shuttling between the cytoplasm and the nucleus. As can be seen from FIG. 2, the amount of NP was significantly decreased by p-cymene after 7, 10 and 24 hours, suggesting that p-cymene may inhibit RNP accumulation in the nucleus at an early stage and throughout the rest of the infection.

### Example 3-Synthesis of Compound A

Compound A (structure shown below) is a compound of Formula (I), wherein R₁ is —CH₃, R₂ is H, R₃ is —CH₂CH₃ and R₄ is —CH₂—O—CH₂CH₂CH₃.

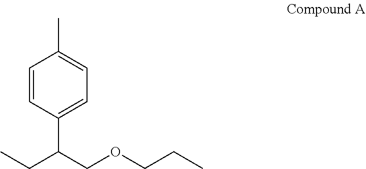

Compound A was synthesized as follows:

Step 1

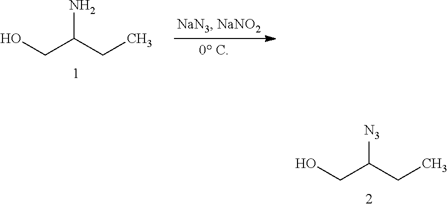

Compound 1 (2-amino-1-butanol) was obtained from Sigma Aldrich, Code: A43804 ALDRICH.

An aqueous solution of NaNO₂ (0.269 g, 3.9×10⁻³ mol of NaNO₂ in 2.2 ml of H₂O) was added to a solution of 2-amino-1-butanol (1) (0.058 g, 0.65×10⁻³ mol) in TFA (10 ml) in a round bottom flask at 0° C. The flask was sealed with a stopper and the reaction solution was stirred for 10 minutes at 0° C.

An aqueous solution of NaN₃ (0.507 g, 7.8×10⁻³ mol of NaN₃ in 2.2 ml of H₂O) was then added, after which the flask, sealed with a stopper, was stirred for 60 minutes at 0° C.

H₂O and then CH₂Cl₂ were then carefully poured in the mixture in order to dilute the TFA. After extraction with CH₂Cl₂/NaHCO₃, the solvents were evaporated under reduced pressure and the product was purified by column chromatography (SiO₂, CH₂Cl₂:EtOH 2%) to obtain compound 2.

Step 2

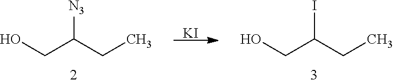

A solution of KI in EtOH (0.996 g, 6.0×10⁻³ mol of KI in 2.5 ml of EtOH) was added to a solution of 2 (0.069 g, 0.60×10⁻³ mol) in EtOH (2.5 ml) in a round bottom flask, followed by one drop of HCl 37% w/w. The reaction solution was heated with reflux and stirred for 60 minutes.

H₂O and then CH₂Cl₂ were then carefully poured in the mixture in order to dilute the reaction solution. After extraction with CH₂Cl₂/NaHCO₃, solvents were then evaporated under reduced pressure and the product was purified by column chromatography (SiO₂, CH₂Cl₂:EtOH, 0.5%) to obtain compound 3.

Step 3

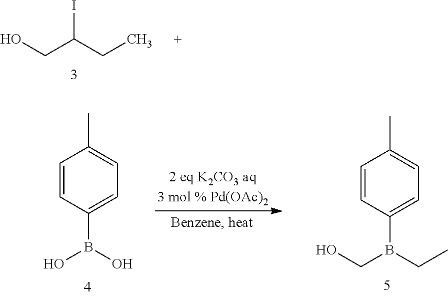

Compound 4 (p-tolylboronic acid) was obtained from Sigma Aldrich, Code: 393622 ALDRICH.

Compound 3 (0.200 g, 1.0×10⁻³ mol), p-tolylboronic acid (4) (0.136 g, 1.1×10⁻³ mol), palladium (II) acetate (3 mg), tetrabutylammonium bromide (0.322 g, 1.0×10⁻³ mol), and potassium carbonate (0.345 g, 2.5×10⁻³ mol) were added to a 5 mL long-neck round-bottom flask, chased by 1.0 mL of distilled water (0.056 mol at 1 g/mL).

A condenser was attached to the flask and capped with a septum, which was pierced with a syringe to provide pressure relief.

The apparatus was heated in a sand bath for 45 minutes at a temperature of approximately 120° C., during which time crystals of product formed in the flask. After cooling to room temperature, the solid product was scraped into a Hirsch funnel for filtration, with the aid of a 10.0 mL wash of hot water (0.56 mol at 1 g/mL). The dried solid was dissolved in 3.0 mL of acetone (0.041 mol at 0.791 g/mL) and the mixture was centrifuged for 2 minutes. Following centrifugation, the supernatant was separated out and boiled down to 2 mL on a sand bath. Crystals were precipitated in the solution using water, at which point the tube was cooled to 0° C., and the crystals were washed and dried to obtain compound 5.

Step 4

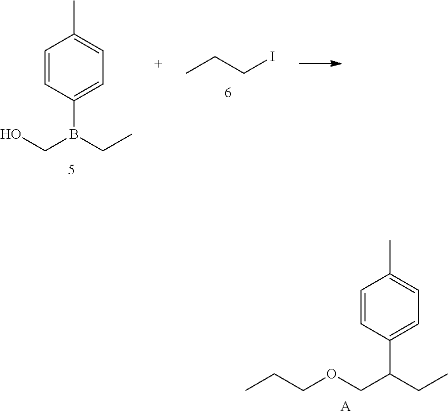

Compound 6 (1-iodopropane) was obtained from Sigma Aldrich, Code: 171883 ALDRICH.

A solution of 1-iodopropane (6) in EtOH (1.019 g, 6.0×10⁻³ mol of 6 in 2.5 ml of EtOH) was added to a solution of 5 (0.098 g, 0.60×10⁻³ mol) in EtOH (2.5 ml) in a round bottom flask, followed by one drop of HCl 37% w/w. The reaction solution was heated with reflux and stirred for 60 minutes.

H₂O and then CH₂Cl₂ were then very carefully poured in the mixture in order to dilute the reaction solution. After extraction with CH₂Cl₂/NaHCO₃, solvents were evaporated under reduced pressure and the product was purified by column chromatography (SiO₂, CH₂Cl₂:EtOH, 0.5%) to obtain Compound A.

### Example 4-In Vitro Testing of Compound A

MDCK-II cells in Minimal Essential Medium (MEM) were seeded into a 6-well plate and incubated at 37° C. The following day, the cells were infected with influenza A/FM/1/47/H1N1 and incubated at 37° C. for 2 hours, followed by washing extensively with serum free MEM.

The infected cells were then cultured in media supplemented with varying concentrations of Compound A at 37° C. for 24 to 48 hours. Supernatants of the infected cells, containing the newly synthesised virions, were harvested and titrated in MDCK-II cells. Upon plaque formation, the infected cells were washed with phosphate-buffered saline (PBS) and fixed with paraformaldehyde. Cell monolayers were stained with crystal violet and observed microscopically.

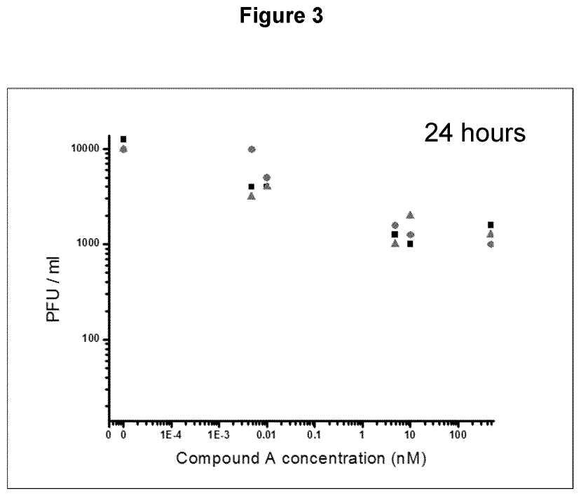

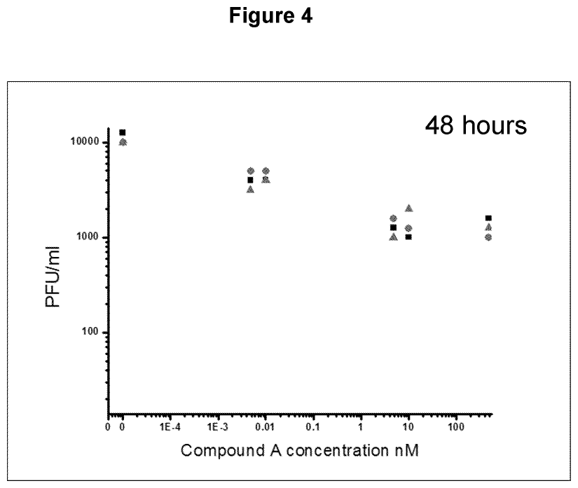

The results are shown in FIGS. 3 and 4, which show the results after 24 hours and 48 hours respectively.

As can be seen from FIGS. 3 and 4, at 24 and 48 hpi (hours post infection), cells treated with Compound A produced fewer viral particles compared to the infected cells, resulting in a significantly decreased virus titer.

### Example 5-In Vitro Testing of Compound A

MDCK-II cells were plated in 12-well plates. After overnight incubation, the cells were washed with phosphate-buffer saline (PBS) and infected with influenza virus A/FM/1/47/H1N1) for 2 hours at 37° C.

The inoculum was then removed and the cells were incubated with MEM containing various concentrations of Compound A for 12 hours. Nuclear RNA was extracted and Real-Time PCR analysis revealed that in the presence of Compound A, the viral M1 genome copy numbers were decreased.

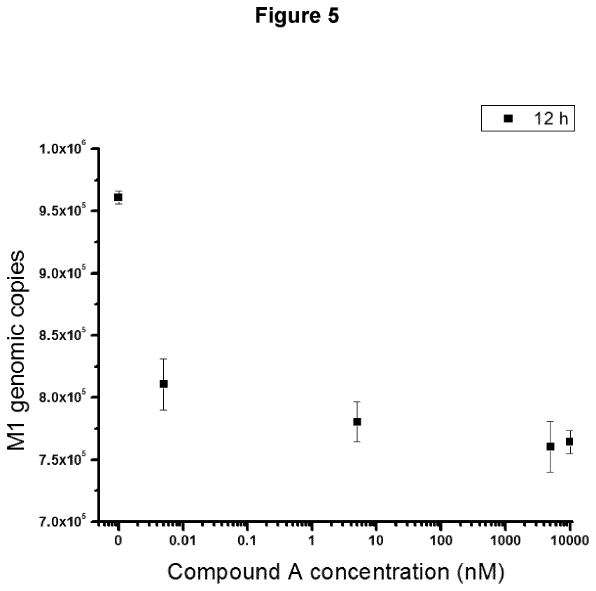

The results are shown in FIG. 5.

As can be seen from FIG. 5, Compound A inhibits (at 12 hours) the synthesis of new viral RNA at all concentrations tested.

The conclusion of these in vitro studies shows that Compound A has a significant anti-viral action, comparable with that of ribavirin. The mechanism of action relies on the inhibition of nucleoprotein nuclear translocation.

### Example 6-in Silico Testing of Compound A

Molecular simulations with Compound A revealed that the binding site on the nucleoprotein molecule is between the RNA and the importin complex binding sites of the molecule. Additional simulations revealed that Compound A binding on the nucleoprotein inhibits the RNA binding, while it has no effect on the importin complex binding. The nucleoprotein may therefore translocate into the nucleus in a non-RNA binding form, depleting the infected cell from its transport mechanism.

In silico testing was also carried out to show that the proposed mechanism can be extended to other RNA-NP viruses. For example, in silico testing shows that Compound A binds to nucleoprotein at sites with similar properties and analogous amino acid sequence for rabies NP (PDB code 2GGT), Ebola (PDB code 5Z9VV) and the novel coronavirus 2019-nCoV Wuhan proteins YP_009724397.2 and YP 009724389.1.

Finally, molecular docking of p-cymene and Compound A were performed on the nucleocapsid SARS-CoV-2 protein (NC), revealing a high affinity binding of compounds at the C-terminal part of the molecule (CTP). Interestingly, this part, and especially the p-cymene and compound A binding amino-acids are in the nuclear localization signal (NLS) of the protein, while their binding inhibits the association of the protein with importin A, and impairs its nuclear translocation. It should be noted that viral RNA binds to the N-terminal part (NTP) of the protein and its association is not modified. Molecular dynamics (1 μs) of the NC protein with Importin A, in the absence or the presence of p-cymene revealed that the agent is able to destabilise the nucleocapsid-importin complex.

### Example 7-In Vitro Testing of p-Cymene and Compound a in SARS-CoV-2 Infected Cells

VERO E6 cells were plated in 12-well plates. After overnight incubation, the cells were washed with phosphate-buffer saline (PBS) and infected with SARS-CoV-2 (isolate 30-287), obtained through culture in Vero E6 cells, from an infected patient.

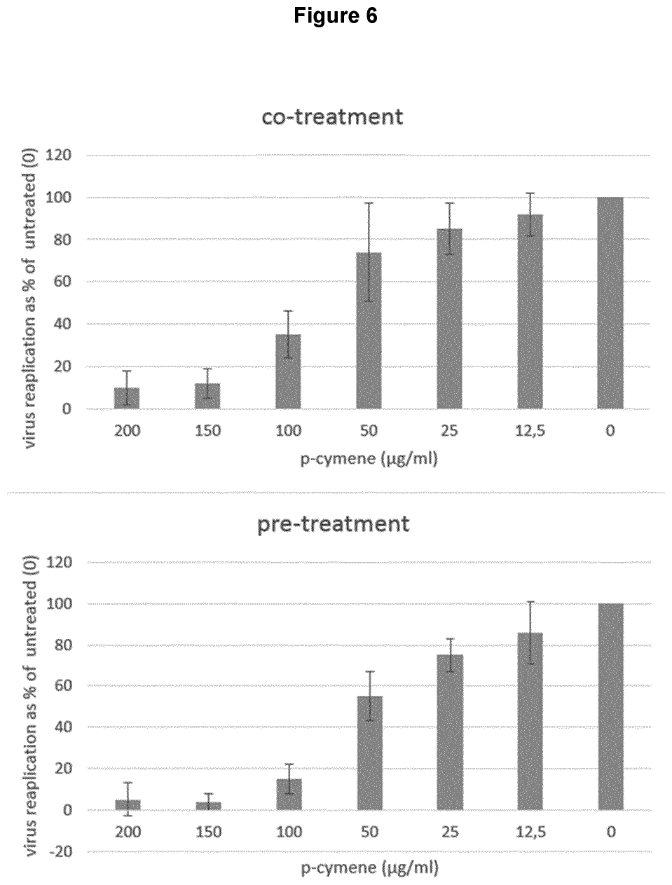

The inoculum was then removed and the cells were incubated with MEM containing various concentrations of p-cymene or Compound A for 96 hours. Nuclear RNA was extracted and Real-Time PCR analysis of viral transcripts is presented in FIG. 6.

In another setting, cells were pre-treated with different concentrations of p-cymene or Compound A for 2 hours before infection with SARS-CoV-2 (isolate 30-287) and incubation of infected cells (in the presence of compounds) was continued for 96 hours, as above. At the end of the incubation, supernatants were assayed for the presence of viral RNA, with quantitative PCR. Results for p-cymene are also presented in FIG. 6. Compound A presented a similar potency.

As can be seen from FIG. 6, p-cymene inhibits (at 96 hours) the synthesis of new viral RNA with an estimated IC₅₀ of 83 μg/ml (co-incubation) and 59 μg/ml (pre-incubation). Similar results were obtained for Compound A (IC₅₀=38 μg/ml (co-incubation) and 9 μg/ml (pre-incubation)).

### Embodiments

The invention also provided the following embodiments:

Embodiment 1. A compound of Formula (I), or a pharmaceutically acceptable salt, solvate, tautomer or isomer thereof, for use in the treatment or prevention of a disease caused by an RNA virus;

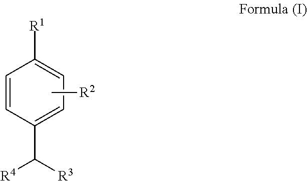

wherein:

R¹ is selected from —OH, C₁₋₆ alkyl which is optionally substituted by one or more —OH group, or C₁₋₆ alkoxy which is optionally substituted by one or more —OH group;

R² is selected from —H, halo, —OH, —NH₂, C₁₋₆ alkyl which is optionally substituted by one or more —OH and/or —NH₂ group, and a 5-membered heterocycle, which is optionally substituted by one or more C₁₋₆ alkyl group, each C₁₋₆ alkyl group being optionally substituted by one or more —OH and/or —NH₂ group;

R³ is selected from —C₁₋₆ alkyl which is optionally substituted by one or more —OH group; and

R⁴ is selected from —C₁₋₆ alkyl which is optionally substituted by one or more —OH group, and —C₁₋₆ alkylene-C₁₋₆ alkoxy which is optionally substituted by one or more —OH group.

Embodiment 2. A compound for use according to embodiment 1, wherein R¹ is selected from —OH, C₁₋₆ alkyl which is optionally substituted by —OH, or C₁₋₆ alkoxy which is optionally substituted by —OH; preferably wherein R¹ is selected from —OH, C₁₋₄alkyl which is optionally substituted by —OH, and C₁₋₄alkoxy which is optionally substituted by —OH.

Embodiment 3. A compound for use according to any preceding embodiment, wherein R¹ is selected from —OH, C₁₋₄ alkyl which is optionally substituted by —OH, and C₁₋₄alkoxy which is optionally substituted by —OH; preferably wherein R¹ is selected from —OH, —CH₃, —CH₂CH₃, CH₂OH, and —O—CH(OH)CH₃.

Embodiment 4. A compound for use according to any preceding embodiment, wherein R² is selected from —H, —Cl, —NH₂, 01-6 alkyl which is optionally substituted by —OH and/or —NH₂, and triazole, which is optionally substituted by C₁₋₆ alkyl which is optionally substituted by —OH and/or —NH₂; preferably wherein R² is selected from —H, —Cl, —NH₂, C₁₋₄ alkyl which is optionally substituted by —OH and/or NH₂, and triazole, which is substituted by C₁₋₄alkyl substituted by —OH and/or NH₂.

Embodiment 5. A compound for use according to any preceding embodiment, wherein R² is selected from —H, —Cl, —NH₂, 01-2 alkyl which is optionally substituted by —OH and/or NH₂, and triazole, which is substituted by C₁₋₄alkyl substituted by —OH and/or NH₂; preferably wherein R² is selected from —H, —Cl, —NH₂, —CH₃, CH(OH)NH₂, and -triazole-CH(OH)(NH₂).

Embodiment 6. A compound for use according to any preceding embodiment, wherein R³ is selected from —C₁₋₆ alkyl which is optionally substituted by —OH; preferably wherein R³ is selected from —O₁₋₄ alkyl which is optionally substituted by —OH.

Embodiment 7. A compound for use according to any preceding embodiment, wherein R³ is selected from —C₁₋₂ alkyl which is optionally substituted by —OH; preferably wherein R³ is selected from —CH₃, —CH₂CH₃ and —CH₂OH.

Embodiment 8. A compound for use according to any preceding embodiment, wherein R⁴ is selected from —C₁₋₆ alkyl which is optionally substituted by —OH, and 6 alkylene-C₁₋₆ alkoxy which is optionally substituted by —OH; preferably wherein R⁴ is selected from —C₁₋₄ alkyl which is optionally substituted by —OH, and —C₁₋₄ alkylene-C₁-₄ alkoxy which is optionally substituted by —OH.

Embodiment 9. A compound for use according to any preceding embodiment, wherein R⁴ is selected from —C₁₋₂ alkyl which is optionally substituted by —OH, and 2 alkylene-C₁₋₄ alkoxy which is optionally substituted by —OH; preferably wherein R⁴ is selected from —CH₂OH, —CH₂—O—CHs and —CH₂—O—CH₂CH₂CH₃.

Embodiment 10. A composition comprising a compound of Formula (I) as defined in any preceding embodiment, or a pharmaceutically acceptable salt, solvate, tautomer or isomer thereof, and a pharmaceutically acceptable carrier.

Embodiment 11. A compound of Formula (I) as defined in any of embodiments 1 to 9, or a composition according to embodiment 10, for use in therapy.

Embodiment 12. A compound of Formula (I)

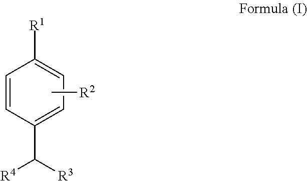

or a pharmaceutically acceptable salt, solvate, tautomer or isomer thereof, wherein:

R¹ is selected from —OH, C₁₋₆ alkyl which is optionally substituted by one or more —OH group, or C₁₋₆ alkoxy which is optionally substituted by one or more —OH group;

R² is selected from —H, halo, —OH, —NH₂, C₁₋₆ alkyl which is optionally substituted by one or more —OH and/or —NH₂ group, or a 5-membered heterocycle, which is optionally substituted by one or more C₁₋₆ alkyl group, each C₁₋₆ alkyl group being optionally substituted by one or more —OH and/or —NH₂ group;

R³ is selected from —C₁₋₆ alkyl which is optionally substituted by one or more —OH group; and

R⁴ is selected from —C₁₋₆ alkyl which is substituted by one or more —OH group, and —C₁₋₆ alkylene-C₁₋₆ alkoxy which is optionally substituted by one or more —OH group;

with the proviso that when R⁴ is —CH₂OH, R² is not H.

Embodiment 13. A composition comprising a compound of Formula (I) as defined in embodiment 12, or a pharmaceutically acceptable salt, solvate, tautomer or isomer thereof, and a pharmaceutically acceptable carrier.

Embodiment 14. A compound according to embodiment 12, or a composition according to embodiment 13, for use in therapy.

Embodiment 15. A compound according to embodiment 12, or a composition according to embodiment 13, for use in the treatment or prevention of a disease caused by an RNA virus.

Embodiment 16. A method of treating or preventing a disease caused by an RNA virus, the method comprising administering to a human or non-human animal a therapeutically effective amount of a compound of Formula (I) as defined in any of embodiments 1 to 9 or 12, or a pharmaceutically acceptable salt, solvate, tautomer or isomer thereof.

Embodiment 17. A compound of Formula (I) as defined in any of embodiments 1 to 9 or 12, or a pharmaceutically acceptable salt, solvate, tautomer or isomer thereof, for use in the manufacture of a medicament for the treatment or prevention of a disease caused by an RNA virus.

Embodiment 18. A compound for use as defined in any of embodiments 1 to 9, 15 or 17, or the method of embodiment 16, wherein the disease caused by an RNA virus is a disease caused by a virus belonging to Group III, Group IV or Group V of the Baltimore classification system.

Embodiment 19. A compound for use as defined in any of embodiments 1 to 9, 15 or 17, or the method of embodiment 16, wherein the disease caused by an RNA virus is selected from Ebola, SARS, novel coronavirus pneumonia, rabies, common cold, influenza, hepatitis C, West Nile fever, poliomyelitis and measles, preferably Ebola, rabies or influenza.

1. A method of treating or preventing a disease caused by an RNA virus, the method comprising administering to a human or non-human animal a therapeutically effective amount of a composition comprising at least about 50 wt. % of one or more compounds of Formula (I), or a pharmaceutically acceptable salt, solvate, tautomer or isomer thereof; 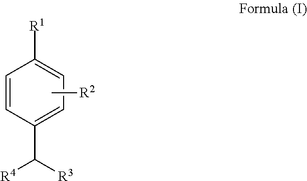
wherein:
R¹ is selected from —OH, C₁₋₆ alkyl which is optionally substituted by one or more —OH group, or C₁₋₆ alkoxy which is optionally substituted by one or more —OH group;
R² is selected from —H, halo, —NH₂, C₁₋₆ alkyl which is optionally substituted by one or more —OH and/or —NH₂ group, and a 5-membered heterocycle, which is optionally substituted by one or more C₁₋₆ alkyl group, each C₁₋₆ alkyl group being optionally substituted by one or more —OH and/or —NH₂ group;
R³ is selected from —C₁₋₆ alkyl which is optionally substituted by one or more —OH group; and
R⁴ is selected from —C₁₋₆ alkyl which is optionally substituted by one or more —OH group, and —C₁₋₆ alkylene-C₁₋₆ alkoxy which is optionally substituted by one or more —OH group.

2. The method according to claim 1, wherein a single compound of Formula (I) constitutes at least about 50 wt. % of the composition, such as at least about 60 wt. %, at least about 70 wt. %, at least about 80 wt. %, at least about 90 wt. %, at least about 95 wt. % or at least about 98 wt. %.

3. The method according to claim 1, wherein the compound of Formula (I) is not

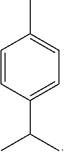

4. The method according to claim 1, wherein R⁴ is selected from —C₁₋₆ alkyl which is substituted by one or more —OH group, and —C₁₋₆ alkylene-C₁₋₆ alkoxy which is optionally substituted by one or more —OH group.

5. The method according to claim 1, wherein R¹ is selected from —OH, C₁₋₆ alkyl which is optionally substituted by —OH, or C₁₋₆ alkoxy which is optionally substituted by —OH; preferably wherein R¹ is selected from —OH, C₁₋₄ alkyl which is optionally substituted by —OH, and C₁₋₄ alkoxy which is optionally substituted by —OH.

6. (canceled)

7. The method according to claim 1, wherein R² is selected from —H, —CI, —NH₂, C₁₋₆ alkyl which is optionally substituted by —OH and/or —NH₂, and triazole, which is optionally substituted by C₁₋₆ alkyl which is optionally substituted by —OH and/or —NH₂; preferably wherein R² is selected from —H, —CI, —NH₂, C₁₋₄ alkyl which is optionally substituted by —OH and/or NH₂, and triazole, which is substituted by C₁₋₄ alkyl substituted by —OH and/or NH₂.

8. (canceled)

9. The method composition for use according to claim 1, wherein R³ is selected from —C₁₋₆ alkyl which is optionally substituted by —OH; preferably wherein R³ is selected from —C₁₋₄ alkyl which is optionally substituted by —OH.

10. (canceled)

11. The method according to claim 1, wherein R⁴ is selected from —C₁₋₆ alkyl which is optionally substituted by —OH, and —C₁₋₆ alkylene-C₁₋₆ alkoxy which is optionally substituted by —OH; preferably wherein R⁴ is selected from —C₁₋₄ alkyl which is optionally substituted by —OH, and —C₁₋₄ alkylene-C₁₋₄alkoxy which is optionally substituted by —OH.

12. (canceled)

13. The method according to claim 1, wherein the compound(s) of Formula (I) is selected from the group consisting of:

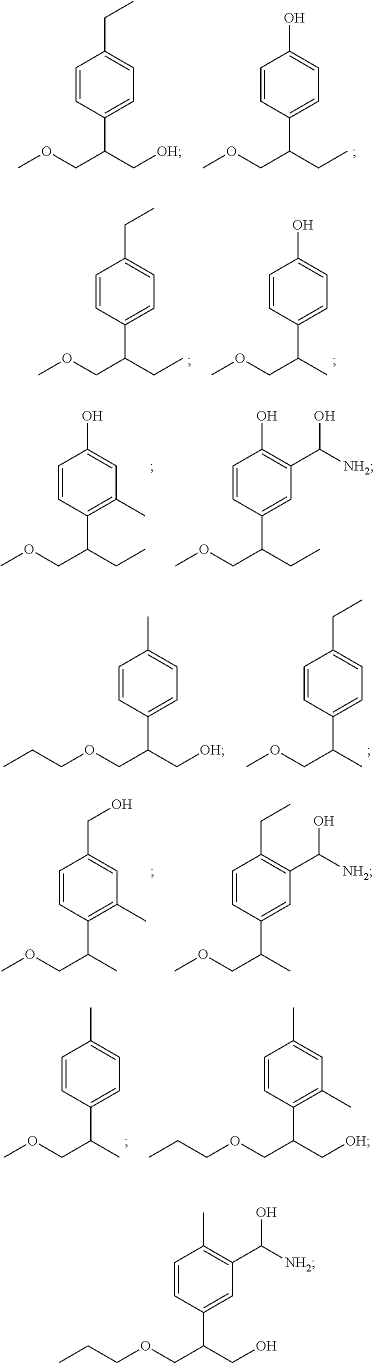

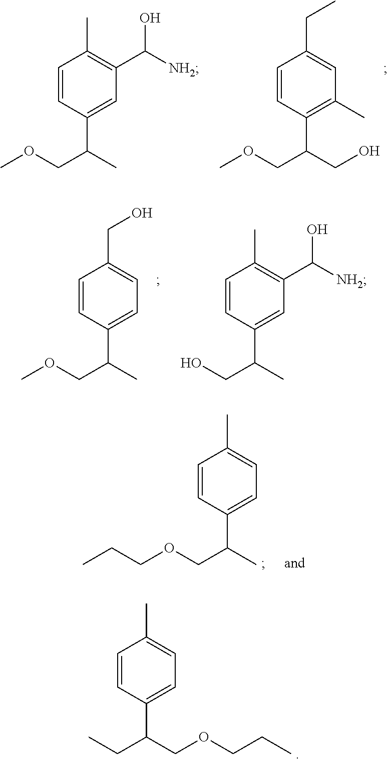

14. The method according to claim 13, wherein the compound(s) of Formula (I) is selected from the group consisting of:

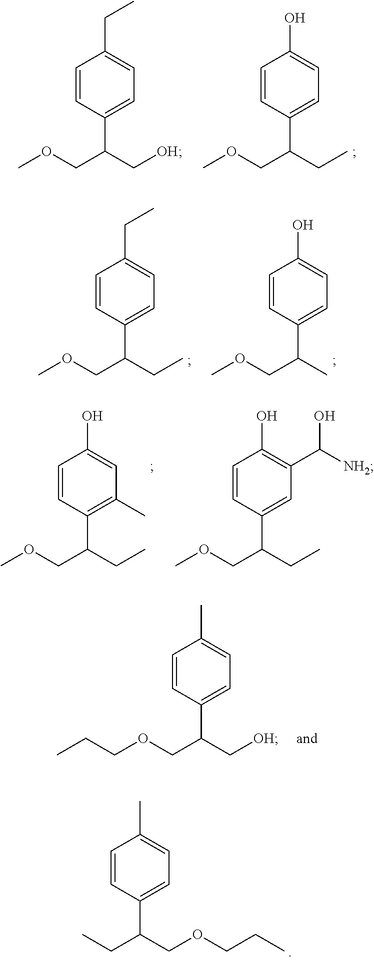

15. The method according to claim 14, wherein the compound of Formula (I) is:

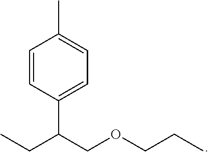

16. A compound of Formula (I) the compound is selected from the group consisting of:

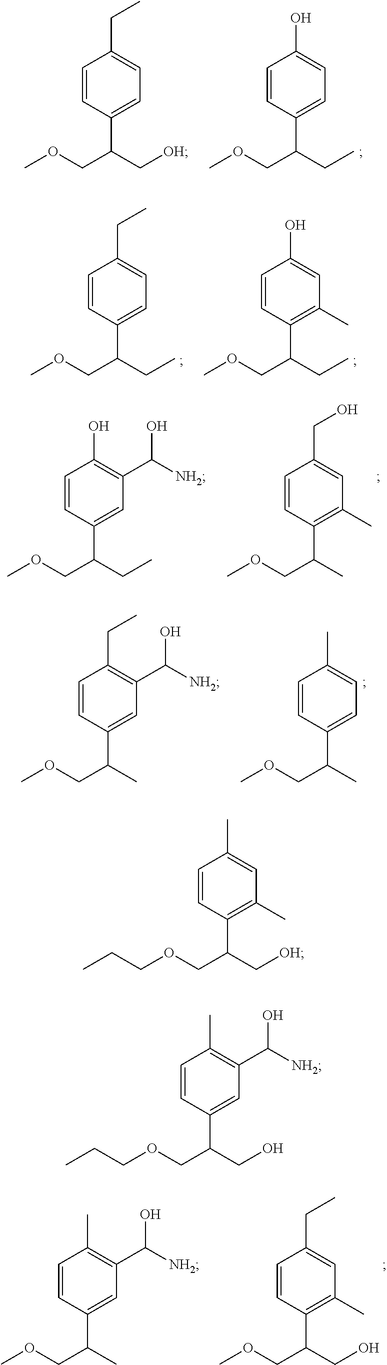

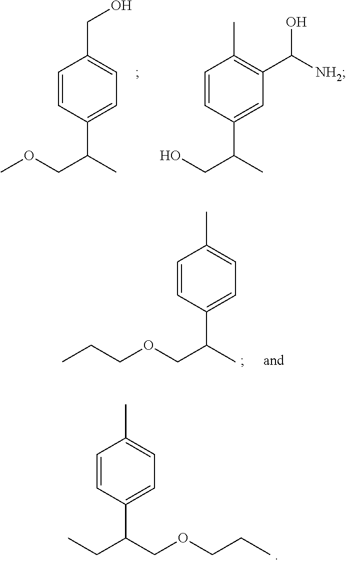

17. The compound according to claim 16, wherein the compound is selected from the group consisting of:

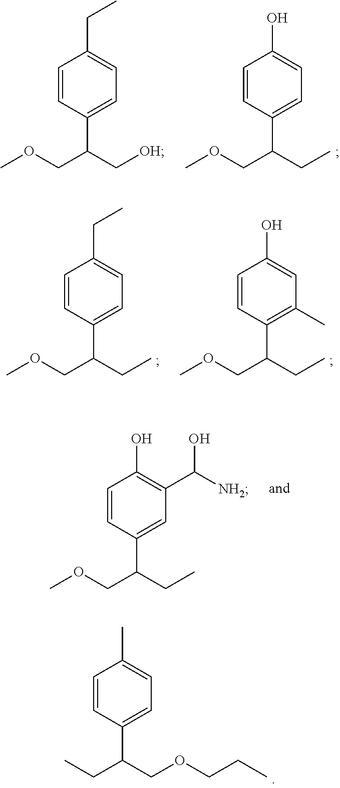

18. The compound according to claim 17, wherein the compound is:

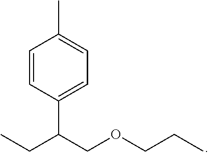

19. A composition comprising one or more compounds of Formula (I) as defined in claim 16, or a pharmaceutically acceptable salt, solvate, tautomer or isomer thereof, and optionally also a pharmaceutically acceptable carrier.

20. The composition according to claim 19, wherein the composition comprises a single compound of Formula (I) as defined in claim 1.

21-26. (canceled)

27. The method of claim 1 wherein the disease caused by an RNA virus is a disease caused by a virus belonging to Group III, Group IV or Group V of the Baltimore classification system.

28. The method of claim 1, wherein the disease caused by an RNA virus is selected from Ebola, SARS, SARS-CoV-2, rabies, common cold, influenza, hepatitis C, West Nile fever, poliomyelitis and measles.

29. The method of claim 1 wherein the disease caused by an RNA virus is selected from SARS-CoV-2, Ebola, rabies or influenza, preferably SARS-CoV-2.

30. The method of claim 1, wherein the disease caused by an RNA virus is SARS-CoV-2.

31-32. (canceled)
